(19) United States
(12) Patent Application Publication
(10) Pub. No.: US 2021/0100608 A1
(43) Pub. Date: Apr. 8, 2021

(54) METHOD OF CONNECTIVE TISSUE RESTORATION

(71) Applicant: CYPHI LLC, Miami, FL (US)

(72) Inventor: Abhinav GAUTAM, Miami Beach, FL (US)

(21) Appl. No.: 17/061,819

(22) Filed: Oct. 2, 2020

Related U.S. Application Data
(60) Provisional application No. 62/987,390, filed on Mar. 10, 2020.
(60) Provisional application No. 62/910,029, filed on Oct. 3, 2019.

(51) Int. Cl. A61B 18/14; A61B 90/00

(52) U.S. Cl. CPC A61B 18/1477; A61B 2018/00994; A61B 90/37

(57) ABSTRACT

The present application provides methods of restoring connective tissue in a patient, utilizing a combination of imaging techniques and minimally invasive tissue remodeling and regeneration, in combination with regenerative proteins. The methods provide a method for treating pain, mobility constraints, and stiffness issues across the interstitial layer (from head to toe), and in various joints and other areas where connective tissues are present. Also provided are kits for carrying out the various methods described herein.

### CROSS-REFERENCE TO RELATED APPLICATIONS

The present application claims benefit to U.S. Provisional Patent Application No. 62/910,029, filed Oct. 3, 2019, and U.S. Provisional Patent Application No. 62/987,390, filed Mar. 10, 2020, the disclosures of each of which are incorporated by reference herein in their entireties.

### FIELD OF THE INVENTION

The present application provides methods of restoring connective tissue in a patient, utilizing a combination of imaging techniques and minimally invasive tissue remodeling and regeneration, in combination with regenerative proteins. The methods provide a method for treating pain, mobility constraints, and stiffness issues across the interstitial layer (from head to toe), and in various joints and other areas where connective tissues are present. Also provided are kits for carrying out the various methods described herein.

### BACKGROUND OF THE INVENTION

In 2018, a network of fluid-filled spaces surrounded by connective tissue throughout the human body, labeled the interstitium, was discovered. (See, e.g., Benias et al., “Structure and Distribution of an Unrecognized Interstitium in Human Tissues,” Scientific Reports 8:4947 (2018)) Previously thought to be a dense wall of collagen, the interstitium is a fluid-filled highway responsible for ion diffusion and cellular protein transport and the primary source of lymph. It has also been determined to be a potential source for areas of treatment for pain, mobility constraints and stiffness issues. What is needed is a mechanism for resetting and remodeling the intersititum, including connective tissue, to treat and manage a patient's pain and mobility limitations.

### BRIEF SUMMARY OF THE INVENTION

The present invention fulfills these needs, providing methods for treating connective tissues, including scar tissue internally, using a natural and non-surgical treatment to free nerves from entrapment, eliminate stubborn scar tissue and restore the anatomy to its pre-injury state.

In embodiments, provided herein is a method for treating a subepidermal tissue in a patient, comprising: visualizing the tissue beneath a skin surface of the patient; vibrating the tissue beneath the skin surface; locating one or more entrapped nerves within the subepidermal tissue; introducing a probe into the subepidermal tissue at the site of the entrapped nerve; manipulating the subepidermal tissue and/or the entrapped nerve with the probe; and injecting a liquid composition into the subepidermal tissue via the probe at the site of the entrapped nerve.

In further embodiments, provided herein is a method for treating a mesoderm-derived tissue in a patient, comprising: visualizing the mesoderm-derived tissue beneath a skin surface of the patient via dynamic ultrasound imaging and real-time image analysis; vibrating the connective tissue beneath the skin surface; locating one or more entrapped nerves within the connective tissue; introducing a needle probe into the connective tissue at the site of the entrapped nerve; manipulating the tissue and/or the entrapped nerve with the needle probe; and injecting a liquid composition into the connective tissue via the needle probe, the liquid composition comprising one or more regenerative proteins and a buffer.

In still further embodiments, provided herein is a kit for carrying out treatment of connective tissue of a patient, comprising: a liquid composition comprising dextrose, plasma-lyte and one or more placental proteins selected from the group consisting of: Basic fibroblast growth factor (bFGF); Epidermal growth factor (EGF); Granulocyte colony-stimulating factor (GCSF); Platelet-derived growth factor (PDGF-AA); Platelet-derived growth factor (PDGF-BB); Placental growth factor (PLGF); Transforming growth factor alpha (TGF-alpha); Transforming growth factor beta 1 (TGF-β1); interleukin 4 (IL-4); interleukin 6 (IL-6); interleukin 8 (IL-8); interleukin 10 (IL-10); Tissue inhibitor of metalloproteinase (TIMP-1); Tissue inhibitor of metalloproteinase (TIMP-2); Tissue inhibitor of metalloproteinase (TIMP-4); Growth Differentiation Factor (GDF-15); Granulocyte macrophage colony-stimulating factor (GM-CSF); Interferon gamma (IFN-γ); Interleukin 1 alpha (IL1-alpha); Interleukin 1 Beta (IL1-β); Interleukin 1 receptor antagonist (IL-1ra); Interleukin 5 (IL-5); Interleukin 7 (IL-7); Interleukin 12 p40 (IL-12p40); Interleukin 12p70 (IL-12p70); Interleukin 15 (IL-15); Interleukin 17 (IL-17); Interleukin 16 (IL-16); Macrophage colony-stimulating factor (MCSF); Osteoprotegerin (OPG); B lymphocyte chemoattractant (CXCL13) (BLC); Chemokine ligand 1 (CCL1) (I-309); Eotaxin-2; Monocyte chemotactic protein 1 (CCL2) (MCP-1); Monokine induced by gamma interferon (CXCL9) (MIG); Macrophage inflammatory protein 1 alpha (CCL3) (MIP-1α); Macrophage inflammatory protein 1 beta (CCL4) (MIP-1β); Macrophage inflammatory protein 1D (MIP-5, CCL15) (MIP-1d); Regulated on activation, normal T cell expressed and secreted (CCL5) (RANTES); Brain-derived neurotrophic factor (BDNF); Bone morphogenetic protein 5 (BMP-5); Endocrine gland-derived vascular endothelial growth factor (EG-VEGF); Fibroblast growth factor 4 (FGF-4); Keratinocyte growth factor (FGF-7); Growth hormone (GH); Insulin-like growth factor (IGF-I); Insulin-like growth factor binding protein-1 (IGFBP-1); Insulin-like growth factor binding protein-2 (IGFBP-2); Insulin-like growth factor binding protein-3 (IGFBP-3); Insulin-like growth factor binding protein-4 (IGFBP-4); and Insulin-like growth factor binding protein-6 (IGFBP-6); and a needle probe for injecting the liquid composition into the connective tissue and for manipulating an entrapped nerve; and instructions for carrying out the method of treatment of the connective tissue.

### BRIEF DESCRIPTION OF THE FIGURES

FIG. 1A is an ultrasound image of normal tissue in a patient.

FIG. 1B is an ultrasound image of tissue containing sections of fibrosis (scarred and damaged tissue) and fusion of the dermis of the interstitial layer.

FIGS. 2A and 2B show ultrasound images taken using doppler imaging, in accordance with embodiments hereof.

FIGS. 2C-2H show artificial intelligence-based methods for locating entrapped nerves, and visualizing, quantifying and determining the health, density and fluidity of the tissue layers.

FIGS. 21 and 2J show the restoration of a distinguishable channel within interstitial tissue.

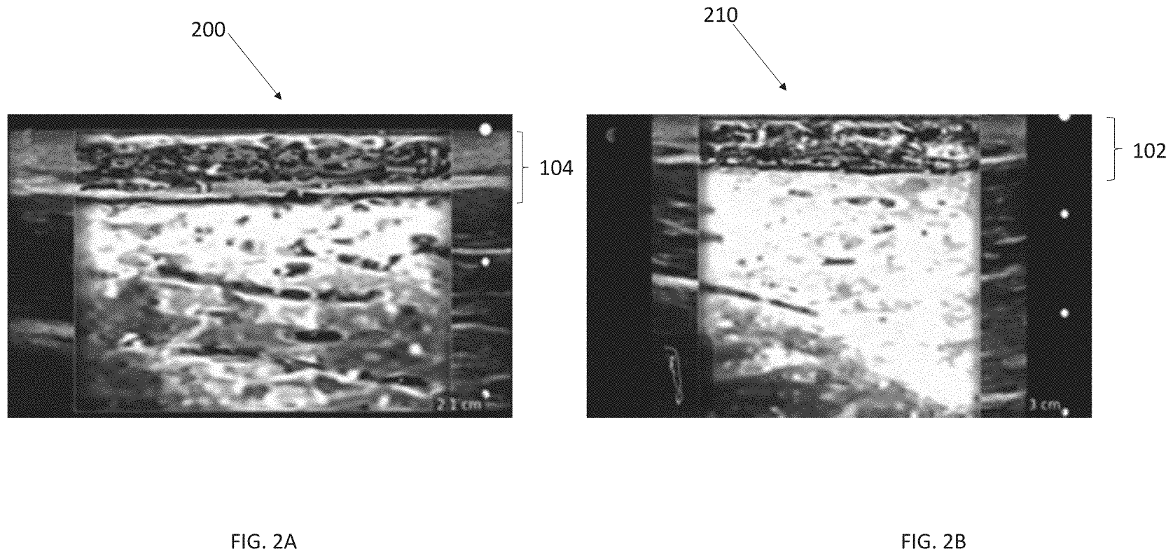

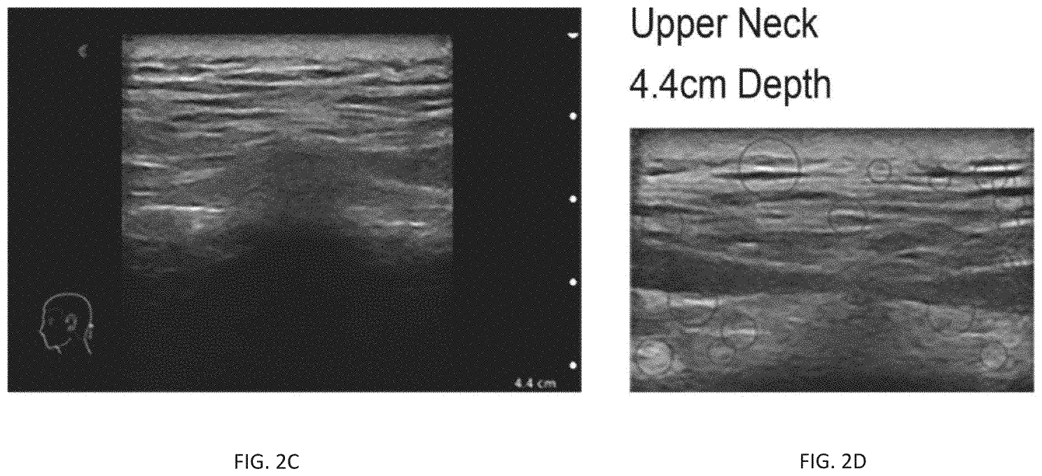

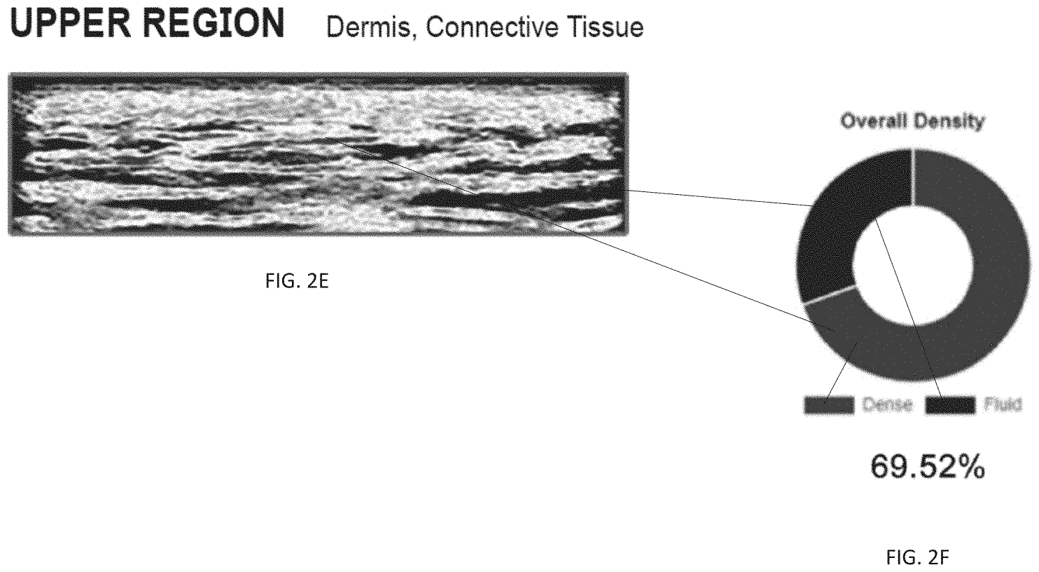

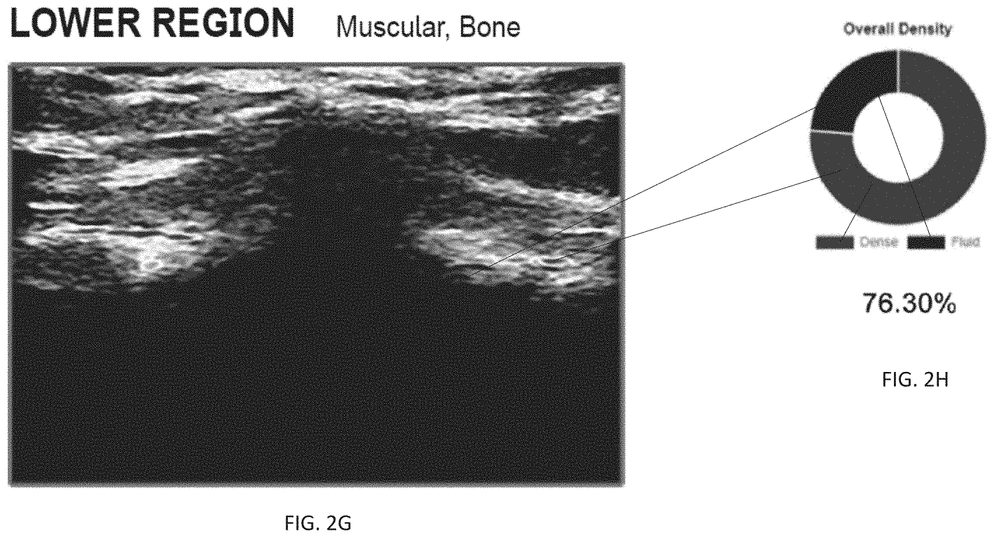

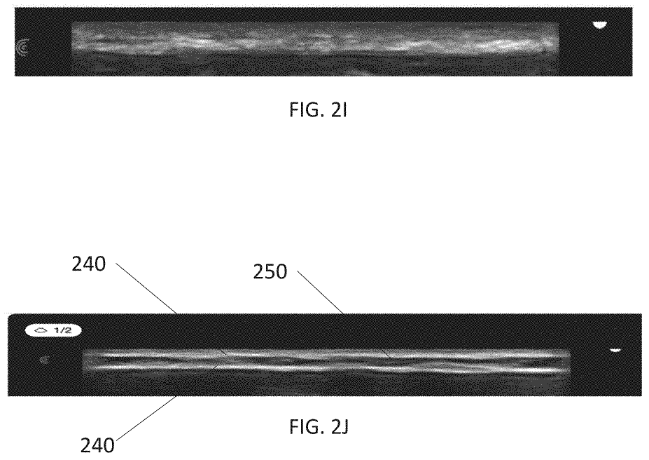

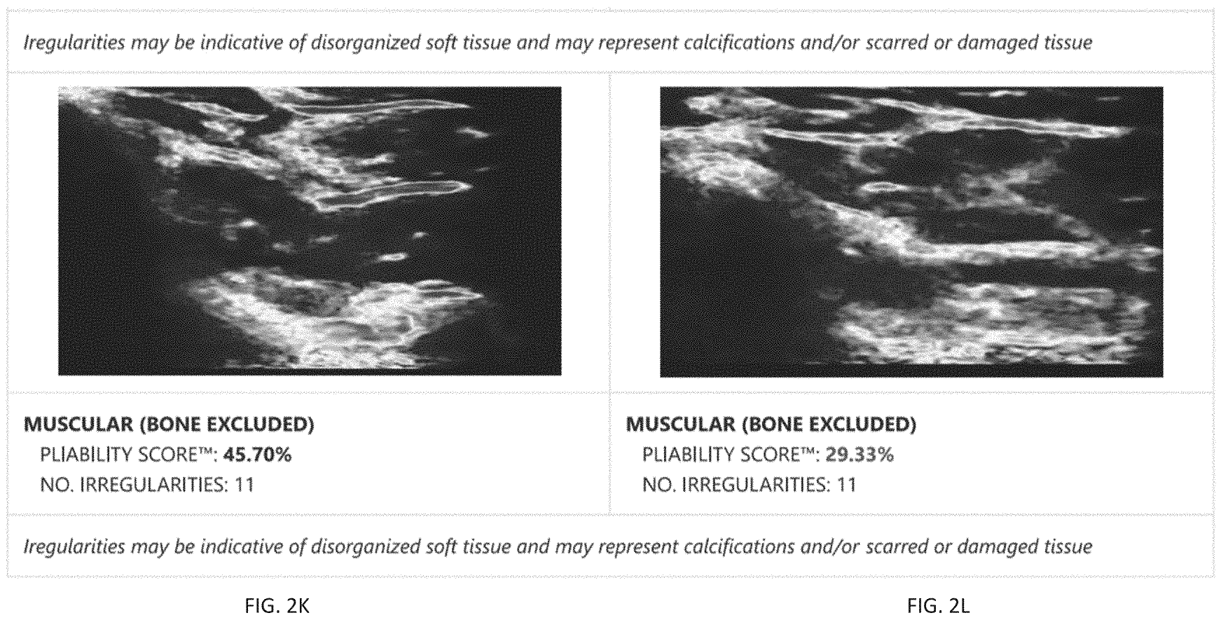

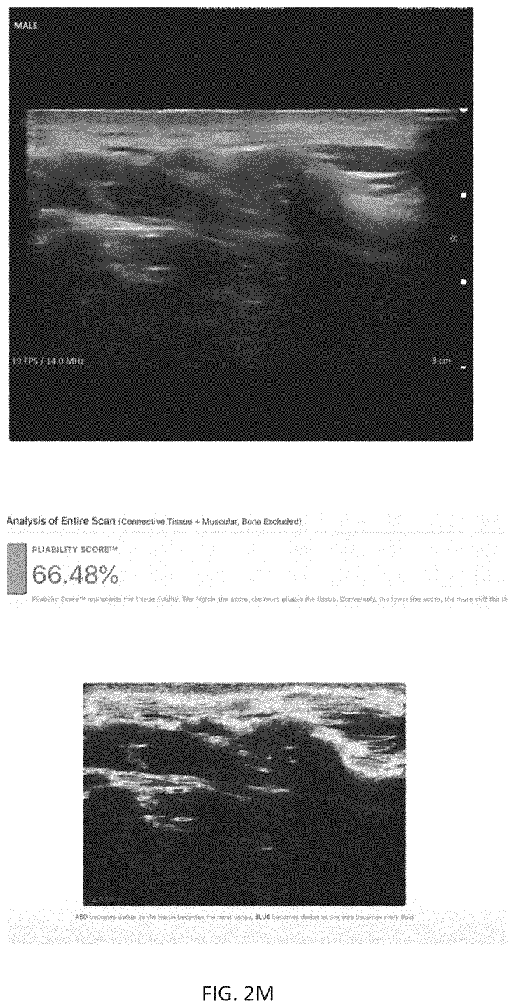

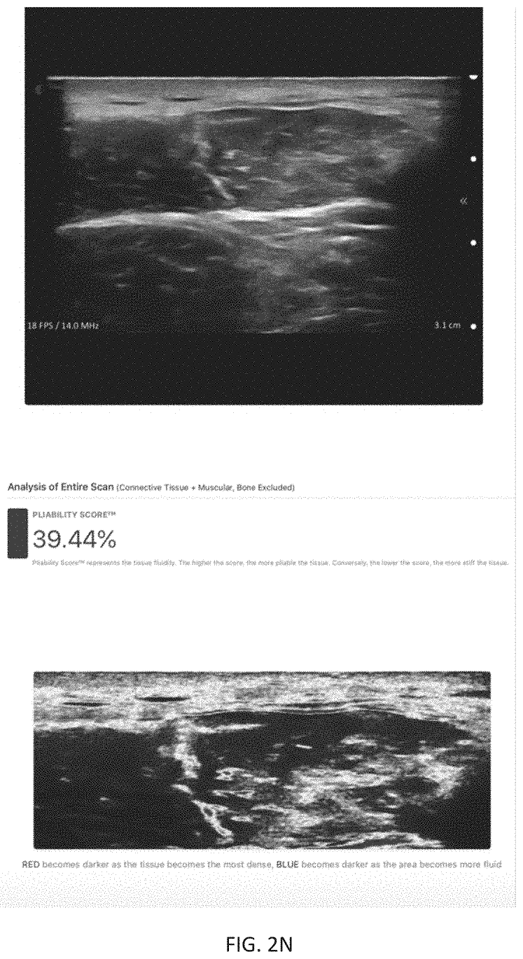

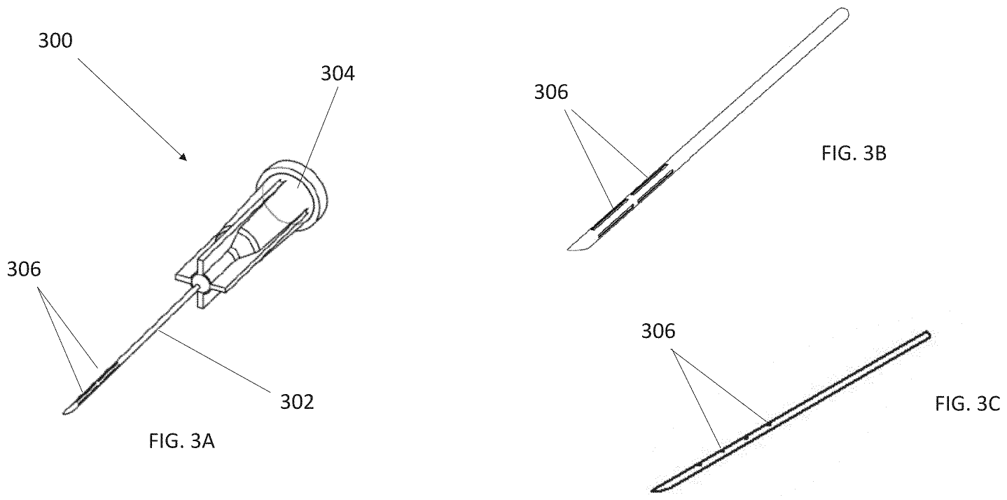

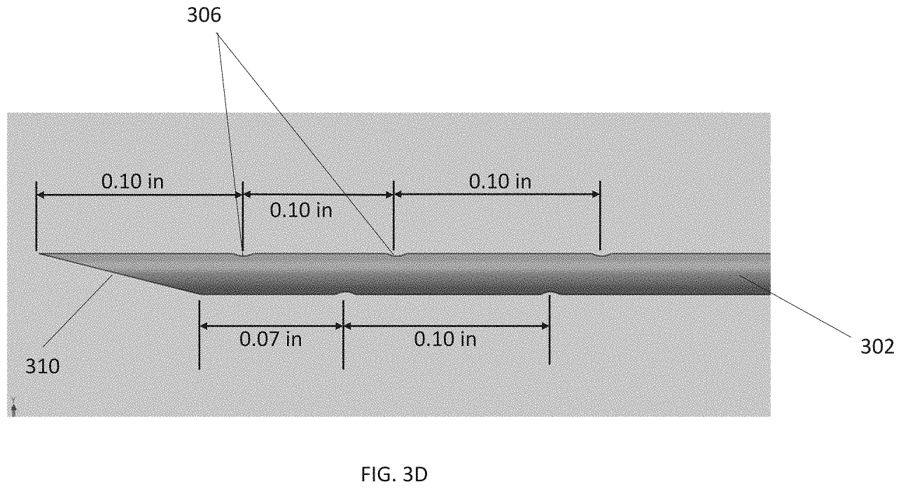

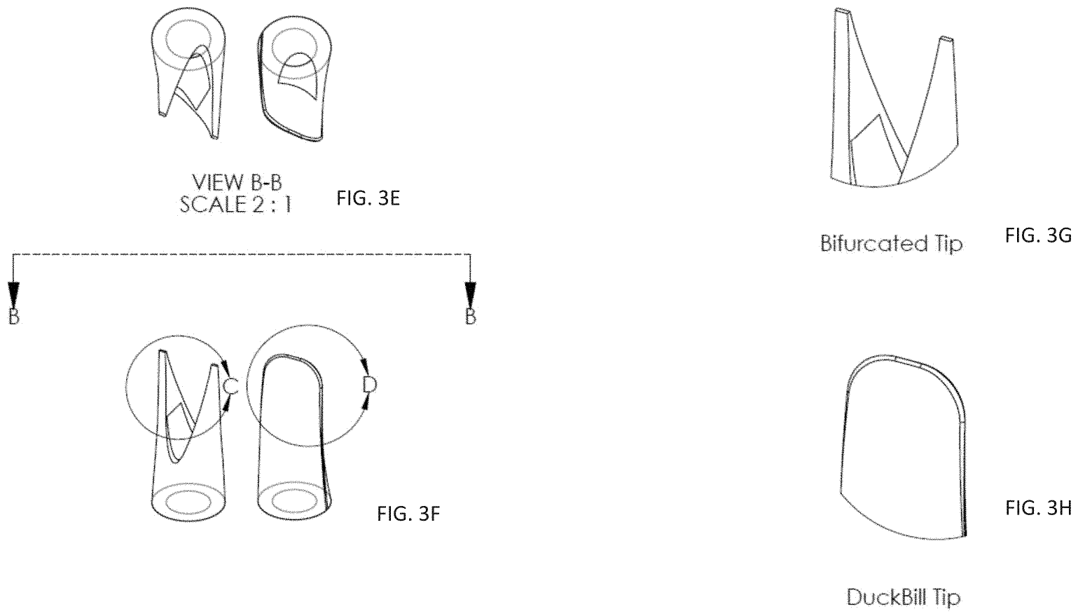

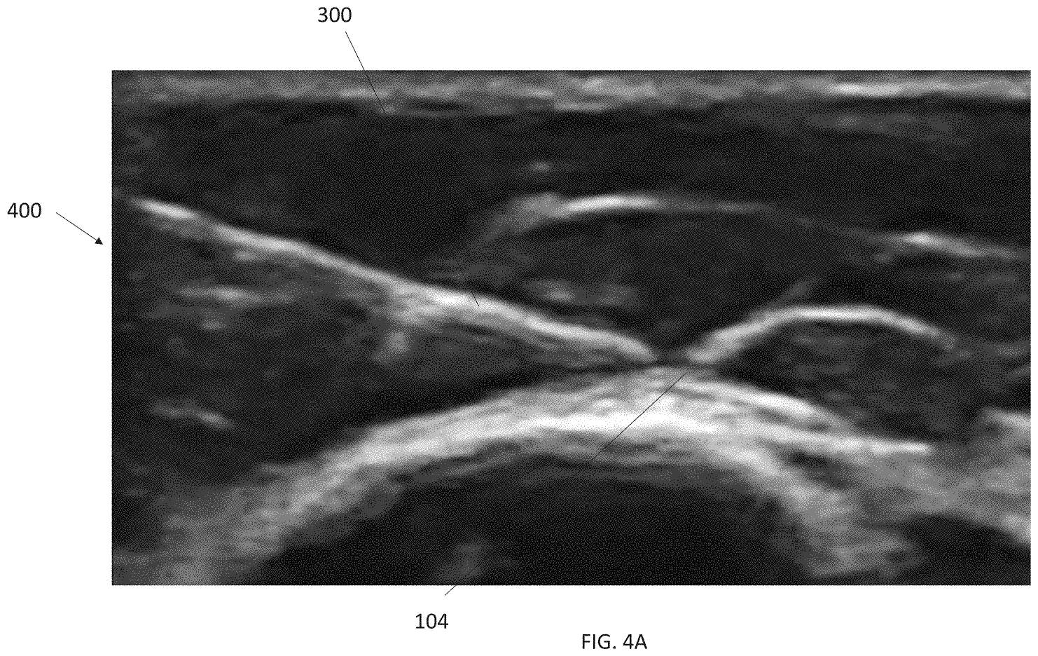

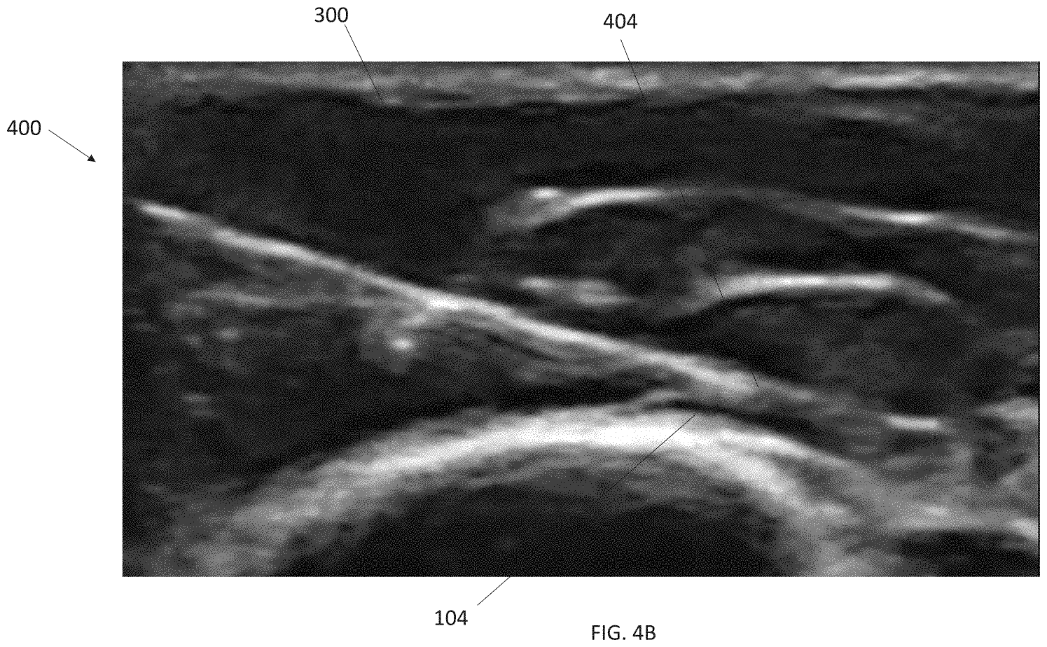

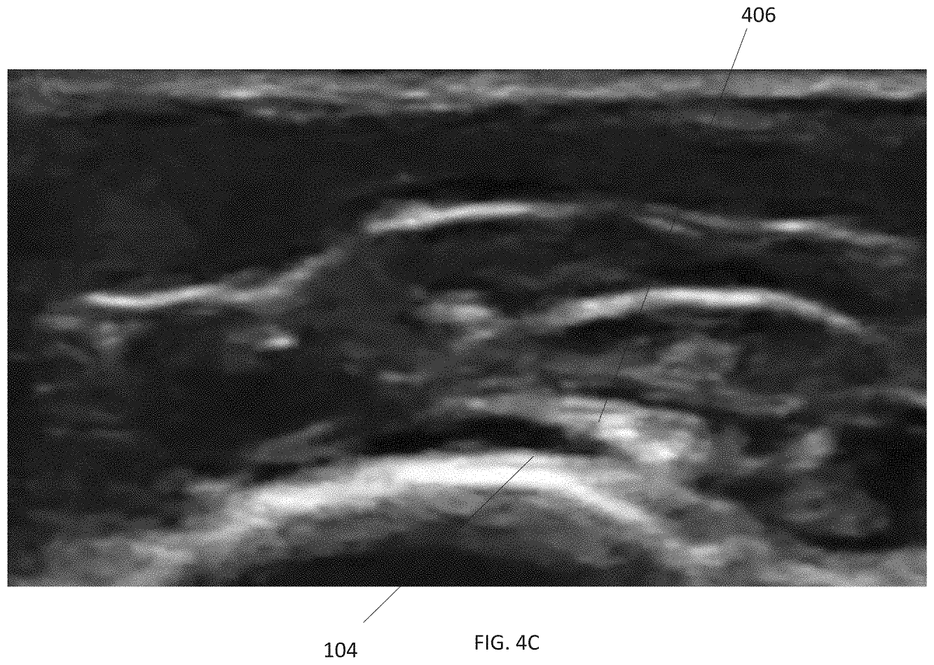

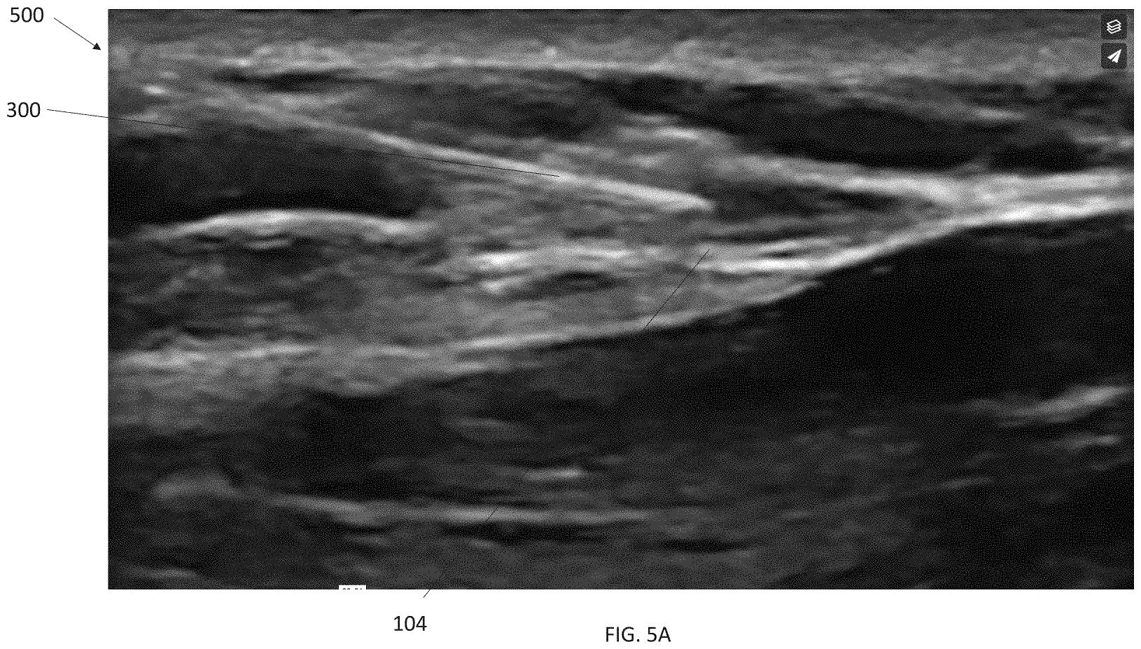

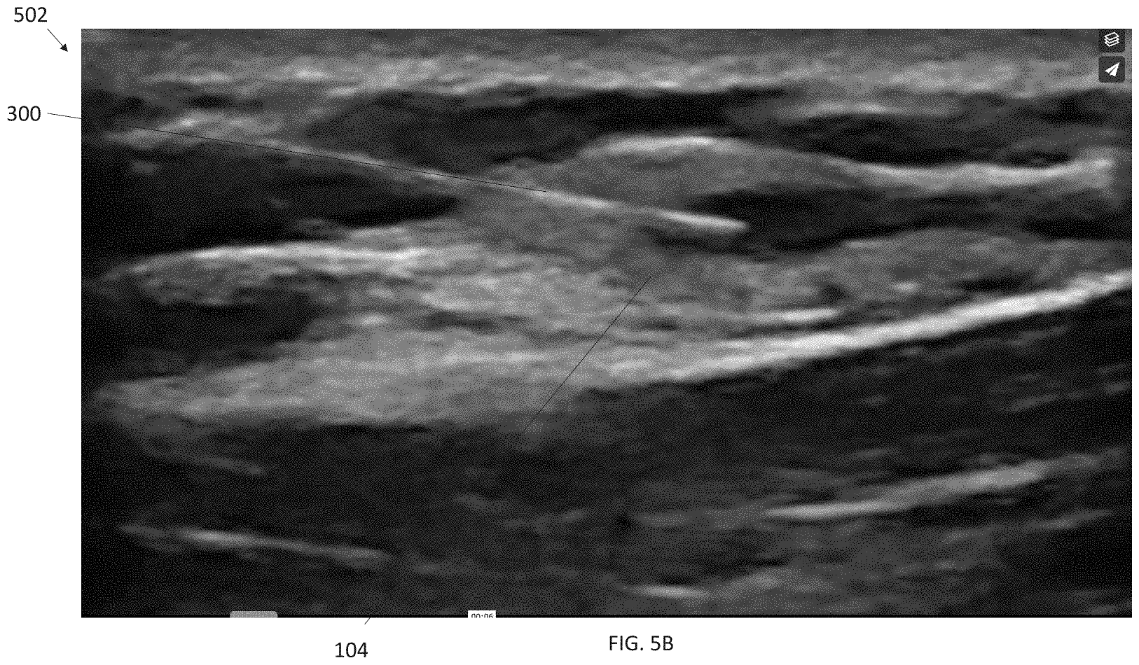

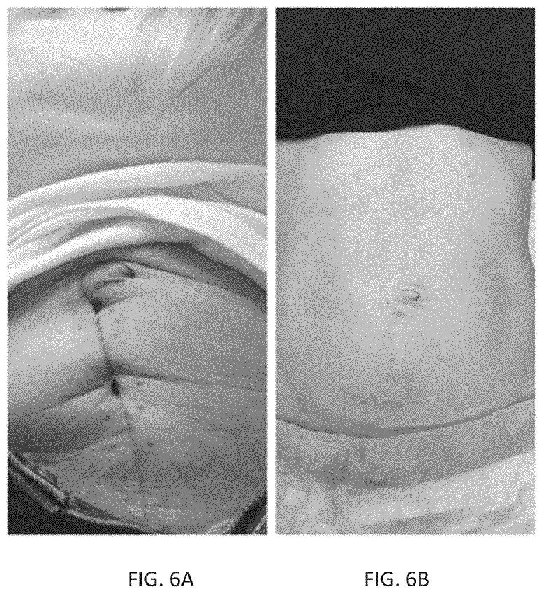

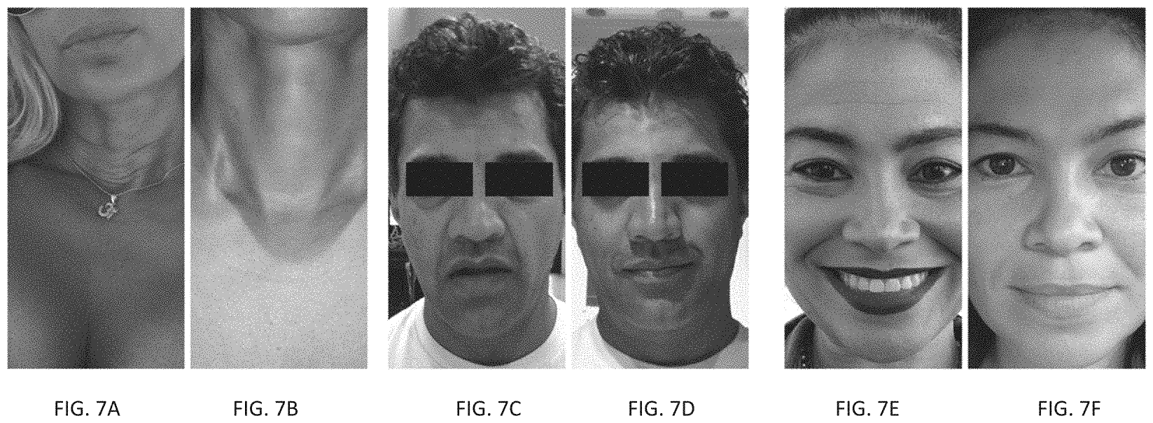

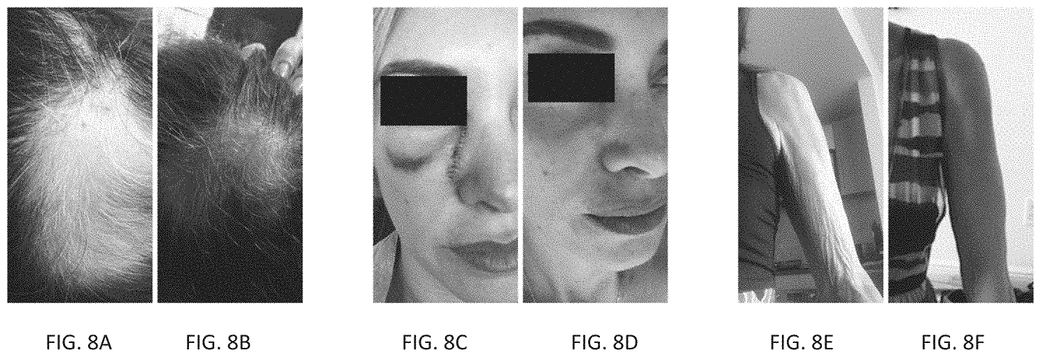

FIGS. 2K-2N show the measurement of the Pliability Score of a tissue.

FIGS. 3A-3D show various probes for use in the embodiments described herein.

FIGS. 3E-3H show exemplary biosculpting tips that can be utilized in the methods and devices described herein.

FIGS. 4A-4C show ultrasound images taken during a procedure as described herein.

FIGS. 5A-5B show ultrasound images taken during a further procedure as described herein.

FIGS. 6A-6B show the effect of remodeling of scar tissue on surface skin.

FIGS. 7A-7F show additional effects of mesenchymal tissue restoration on surface skin.

FIGS. 8A-8F show additional results of the methods of treatment described herein.

FIG. 9 shows the location of connective tissues throughout the body.

### DETAILED DESCRIPTION OF THE INVENTION

It should be appreciated that the particular implementations shown and described herein are examples and are not intended to otherwise limit the scope of the application in any way.

The published patents, patent applications, websites, company names, and scientific literature referred to herein are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As used in this specification, the singular forms “a,” “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Technical and scientific terms used herein have the meaning commonly understood by one of skill in the art to which the present application pertains, unless otherwise defined. Reference is made herein to various methodologies and materials known to those of skill in the art.

In embodiments, provided herein are methods for treating a subepidermal tissue in a patient. The methods described herein have been termed RELIEF® (real-time, echo, located, interventional, epineural, fibrolysis).

As used herein, a “subepidermal tissue” refers to a tissue of a patient that lies below the outer surface of the skin (the epidermis), suitably sits within the interstitium, and is part of the submucosa and a fluid-filled interstitial space, draining to lymph nodes and supported by a complex network of thick collagen bundles. “Subepidermal tissue” also includes tissue within muscle that is scarred or damaged. The methods and procedures described herein are suitably carried out in mammalian patients, including cats, dogs, primates, cows, goats, pigs, etc., and in more suitable embodiments, are carried out in human patients. Human patients include both males and females, and include all age groups from child through adult.

The methods described herein suitably include visualizing the tissue beneath a skin surface of the patient. As used herein “visualizing” the tissue refers to the use of one or more imaging instruments or imaging methods to provide a virtual picture of the tissue below the skin surface, rather than directly cutting into the skin surface to reveal the structure of the tissue. This visualizing can utilize various forms of methods for imaging beneath the skin surface, including x-rays, ultrasound, magnetic resonance imaging, computed tomography, etc.

In exemplary embodiments, the visualizing comprises ultrasound imaging, which includes both 2-dimensional and 3-dimensional ultrasound imaging. Ultrasound imaging is well known in the art and utilizes high-frequency sound waves to produce dynamic visual images of organs, tissues or blood flow inside the body. Generally, the ultrasound imaging instrumentation that is utilized in the methods described herein will include the use of an ultrasound probe that is placed in contact with the surface of the skin to provide and receive the sound waves. An exemplary ultrasound instrumentation includes a linear handheld ultrasound scanner, such as from CLARIUS, Burnaby, Canada. In other embodiments, an ultrasound probe that is placed internal to a patient, for example in a cavity or opening of a patient, can also be used. In suitable embodiments, the ultrasound imaging utilized in the methods provided herein operates at a frequency of about 50 kHz up to about a few GHz. More suitably, the ultrasound imaging operates at a frequency of about 1 MHz-200 MHz, about 4-100 MHz, about 4-50 MHz, about 4-20 MHz, about 20-50 MHz, about 4-15 MHz, or about 5 MHz, about 10 MHz, about 15 MHz, about 20 MHz, about 25 MHz, about 30 MHz, etc.

Suitably, the subepidermal tissue that is visualized in the methods described herein is located within about 20 cm from the surface of the skin. The position of the subepidermal tissue below the skin surface will vary greatly based on the type of the tissue, as well as the location on the patient and the patient's body mass. Suitably, the subepidermal tissue will be located within about 15 cm from the surface of the skin of the patient, more suitably within about 10 cm, within about 9 cm, about 8 cm, about 7 cm, about 6 cm, about 5 cm, about 4 cm, about 3 cm, about 2 cm, or within about 1 cm from the surface of the skin.

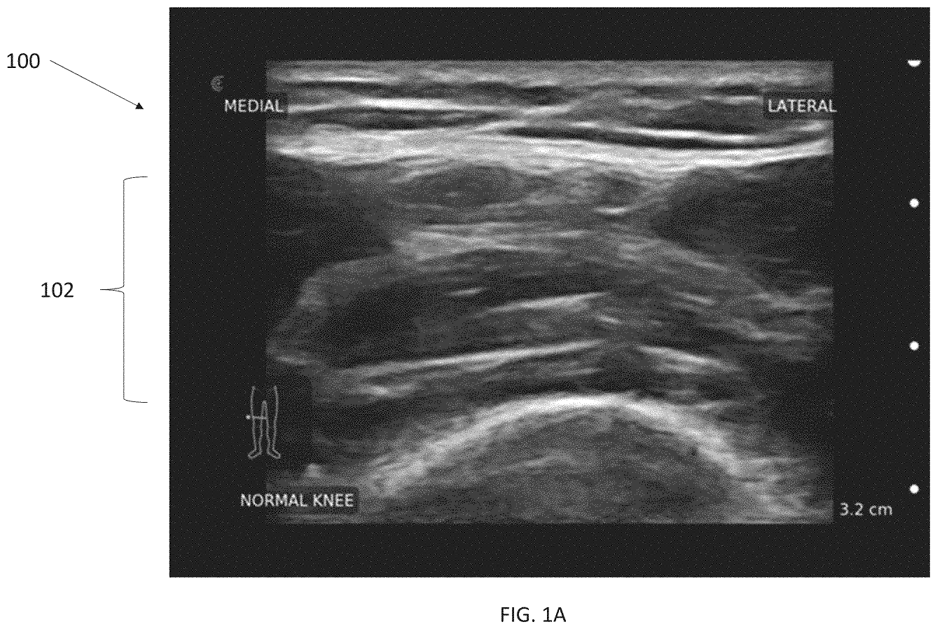

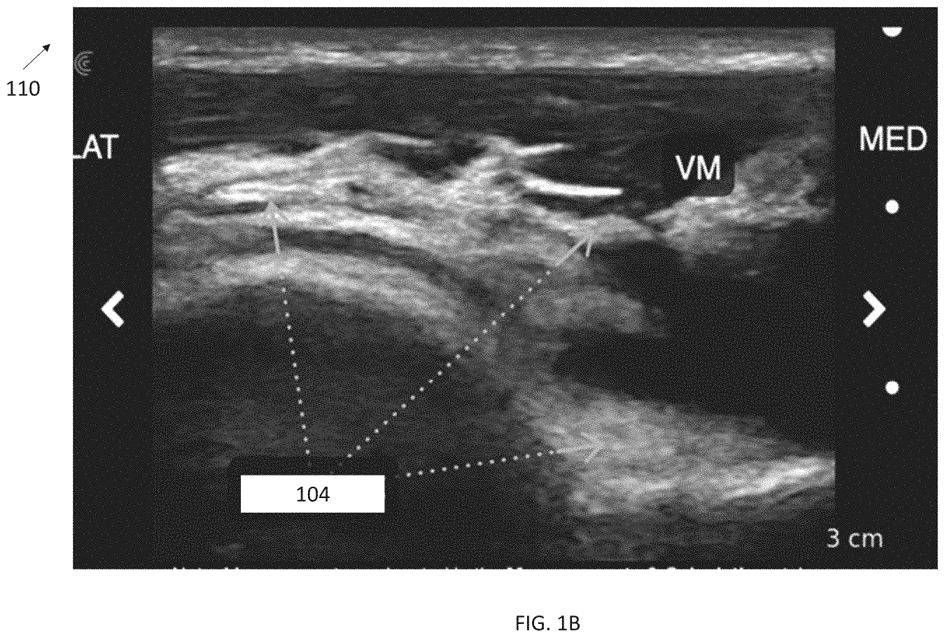

FIG. 1A shows an ultrasound image 100 taken of a normal knee of a human patient. Ultrasound image 100 shows a 2-dimensional view into the patient's knee, with the top of the image representing the skin surface, and the bottom representing approximately 3.2 cm into the patient's knee, below the surface. The medial and lateral sides of the knee are also represented. Ultrasound image 100 illustrates the substantially normal 102 subepidermal tissue, with characteristic basement membranes of each tissue plane neatly gliding over one another in a laminar nature.

In contrast, FIG. 1B shows an ultrasound image 110 taken of a physically compromised knee, illustrating areas of fibrosis 104, represented as dense white tissue in the image 110. The top of the image shows a fusion of the dermis to the interstitial layer. These areas of fibrosis 104 represent areas of the subepidermal tissue that has been blocked, perturbed, scarred, damaged, or otherwise injured, and suggest a neurological compromise. As described herein, it is in these areas that a physical disturbance to a cutaneous nerve or inappropriate amounts of tension can cause hypertrophy, and resulting pain, in the subcutaneous tissue.

The methods described herein suitably further include vibrating the tissue beneath the skin surface to produce greater imaging clarity of the compromised tissues. As shown in FIG. 2A and 2B, doppler ultrasound images have been taken of a physically compromised trapezius (FIG. 2A) and a remodeled trapezius post treatment in accordance with the methods described herein (FIG. 2B). As shown in doppler image 200, areas of fibrosis 104 can be seen close to the surface of the skin (top of the image) and appear as dark sections of tissue. Healthy tissue appears bright in response to the vibration as imaged by doppler ultrasound. Image 210 shown in FIG. 2B, in contrast, shows healthy, remodeled tissue 102 with very little fibrosis, as more of the image appears bright, with only small areas of dark, stiff, dense tissue.

Methods for vibrating the tissue beneath the skin surface include an acoustic radiation force impulse or a palpitation. As used herein an “acoustic radiation force impulse” includes any sound wave generation that provides a force strong enough to vibrate the subepidermal tissue. Acoustic radiation force impulses can be generated, for example, by the same ultrasound equipment and probe that are used to visualize the tissue, or can be from a separate instrument designed to produce an acoustic radiation force. Examples of acoustic radiation include shear-wave elastography, and in embodiments, exemplary characteristics for an acoustic radiation force impulse are provided below:

TABLE 1
Acoustic Radiation Force Impulse Characteristics

| | 2-7 MHz (can be increased | |
|---|---|---|
| | above 10 MHz, including | |
| Frequency | at or above 20 MHz) | |
| F-number | F-2 | |
| Intensity (Iₛₚₚₐ · 5) | 1200-1600 | W/cm² |
| | (suitably 1400 | W/cm²) |
| Intensity (Iₛₚₜₐ · 3) | 0.7 | W/cm² |
| Mechanical Index | 1.5-3.0 | |
| Pulse Duration | 0.1-0.5 | ms |
| Temperature Rise | 0.02-0.2° | C. |
| Displacement (peak) | 10-20 | μm |

Palpitations include manual palpitations (e.g., applying a tapping via the hand or fingers), as well as the use of various massagers or vibrating machines to provide the palpitation.

Various different tissues can be treated using the methods described herein. Suitably, the subepidermal tissue is a mesoderm-derived tissue. As used herein, “mesoderm-derived tissue” refers to a tissue that comes from the mesoderm, one of the three germinal layers present in embryonic development. Mesoderm-derived tissues include one or more of connective tissue, muscle, fat, bone, nerve, tendon, and ligament, etc. In suitable embodiments, the subepidermal tissue treated using the methods described herein is a connective tissue of a patient. As used herein, “connective tissue” refers to tissue that joins or is found between other tissues in the body, and in general, supports and protects the body. Connective tissue includes connective tissue proper, and special connective tissue. Connective tissue proper consists of loose connective tissue and dense connective tissue. Loose and dense connective tissue are distinguished by the ratio of ground substance to fibrous tissue. Loose connective tissue has much more ground substance and a relative lack of fibrous tissue, while the reverse is true of dense connective tissue. Dense regular connective tissue, found in structures such as tendons and ligaments, is characterized by collagen fibers arranged in an orderly parallel fashion, giving it tensile strength in one direction. For example, the methods and techniques described herein can be used to repair ligament damage, such as anterior cruciate ligament (ACL) and medial collateral ligament (MCL) tears. Dense irregular connective tissue provides strength in multiple directions by its dense bundles of fibers arranged in all directions. Special connective tissue consists of reticular connective tissue, adipose tissue, cartilage, bone, and blood. Other kinds of connective tissues include fibrous, elastic, and lymphoid connective tissues.

In further embodiments, the mesoderm-derived tissue that is treated results in remodeling of surface tissue. For example, scar tissue can be remodeled below the skin, resulting in the changes in morphology of the skin covering the scar tissue.

The subepidermal tissue, and in embodiments the connective tissue, that is treated using the methods described herein can be located in any location in a patient's body, including for example, in a patient's neck, back, knees, hips, shoulders, elbows, feet, ankles, toes, hands, wrists, and/or fingers, etc.

The methods of treatment described herein suitably further include locating one or more entrapped nerves within the subepidermal tissue. Various visualization methods, alone or in combination, can be utilized to locate an entrapped nerve. In general, entrapped nerves are often located at the interplanar space, where cutaneous nerves traverse the connective tissue. Situations where the pathway of a nerve is blocked/perturbed/scarred/etc., is suggestive of neurological compromise. Pain is typically generated by a physical disturbance to a cutaneous nerve or mechanoreceptor subjected to inappropriate amounts of tension. This can cause hypertrophy of the connective tissue. Attention is paid to the morphology of the connective tissue plane. Ideally it is patent, laminar, and well hydrated (its appearance is a black horizontal plane in ultrasound images). Increased amounts of dense collagen represent a decrease in the amount of ground substance (bioactive hydrogel) present in local area.

For example, in embodiments, ultrasound visualization of the subepidermal tissue can be used in concert with anatomical information regarding the positioning, depth, and other location information, to provide an actual or virtual representation of the location of one or more entrapped nerves. In embodiments, an actual representation can be obtained by combining, either manually (i.e., by the doctor in real time) or electronically, the ultrasound imaging in combination with a visual representation of the anatomical structure of the subepidermal tissue in real time. This process results in a real-time image analysis method to help locate the entrapped nerves and/or abnormal tissue (e.g., scarred or fibrotic tissue), as well as soft tissue located in the area where treatment is desired, including ligaments, tendons, fat, muscle, skin and cartilage. In embodiments, a three-dimensional, anatomical visual representation of the human body can be used to provide the location and positioning of one or more entrapped nerves, including for example a Complete 3-dimensional Anatomy representation provided by 3D4Medical (see, 3d4medical.com)

In additional embodiments, artificial intelligence or other computerized methods can be utilized to represent a virtual view of the subepidermal tissue, providing a real-time imaging and image analysis method. For example, methods described in U.S. Provisional Patent Application No. 62/787,414, filed Jan. 2, 2019, entitled “Comprehensive Connective Tissue Analysis Methods—Multimodal sonography with AI powered automated assessment/interpretation, and index/scoring of soft tissue (epidermis, dermis, hypodermis, connective tissue, and muscle) for occupational therapy, workman comp, and sports related injury,” (the disclosure of which is incorporated by reference herein in its entirety, including the various computerized methods and algorithms provided therein), can be used in the virtual representation. Additional methods are described in U.S. 62/958,430, “Methods and Computing System for Processing Ultrasound Image to Determine Health of Connective Tissue Layer,” filed Jan. 8, 2020, the disclosure of which is incorporated by reference herein in its entirety.

For example, such artificial intelligence-based methods include the assessment of tissue by the means of image processing and an appropriate form of an artificial intelligence workflow which gives an index or score which ranks and classifies tissue. This is described as a deep learning ultrasound computer aided design (CAD) system along with feature extraction by classification and a deep learning convolutional neural network. Briefly, the steps of artificial intelligence (AI) screening and assessment of connective tissue ultrasound images includes: (i) the candidate regions are first detected by means of image processing techniques; (ii) the candidate regions are represented by a set of features such as morphological or statistical information; and (iii) the features are fed into a classifier, e.g., support vector machine (SVM), to output a probability or make a decision of being diseased, as described in U.S. Provisional Patent Application No. 62/787,414 and U.S. 62/958,430. The methods suitably utilize Corner Classification Neural Networks (CCs), which are a model of short-term memory. Regions are kept separate by the use of unary coding or its generalizations making the generalization more consistent with the manner it is done by the human vision system. The intelligent engine disclosed in U.S. Provisional Patent Application No. 62/787,414 and U.S. 62/958,430 applies CC networks to ultrasound imaging.

FIG. 2C-2H show an exemplary use of the artificial intelligence methods described herein and in U.S. Provisional Patent Application No. 62/787,414 and U.S. 62/958,430 to provide a virtual image of the subepidermal tissue and aid in locating entrapped nerves, as well as visualizing, quantifying and determining the health, density and fluidity of tissue layers. These artificial intelligence methods have been termed SCARMAP®.

FIG. 2C shows a 2-D ultrasound image of the upper neck of a patient. FIG. 2D shows specific areas in the image (circles) that are to be evaluated using the artificial intelligence-based virtual imaging techniques described herein.

FIGS. 2E and 2F illustrate the high density areas located in the upper half of the connective tissue scanned in FIG. 2C, illustrating a high likelihood of scarred tissue. FIGS. 2G and 2H illustrate the density of tissue in the lower half of the ultrasound scan of FIG. 2C, representing the more muscular areas of the tissue.

Using the artificial intelligence methods described herein, images in FIGS. 2E and 2G, high density represents scarring and injuries, areas where stiffness and lack of mobility are found. Lower density areas represent higher fluid levels. In the lower region (FIG. 2G), high density areas represent the likelihood of past injuries. Issues in the lower region can translate to fusions in the upper region over time. The artificial intelligence-based images also show the structure of the connective tissue. Healthy tissue typically shows horizontal borders with easily identified fluid channels. Damaged tissue shows inflammation and fusions, resulting in broken or branching borders.

Textures of the tissue shown in the artificial intelligence-based virtual images in FIGS. 2E and 2G shows the presence of high density in the upper half (FIG. 2E) with a lack of fluid channels and potentially completed fused tissue layers. As overall density increases, a spackled texture is seen, representing the presence of possible fusions.

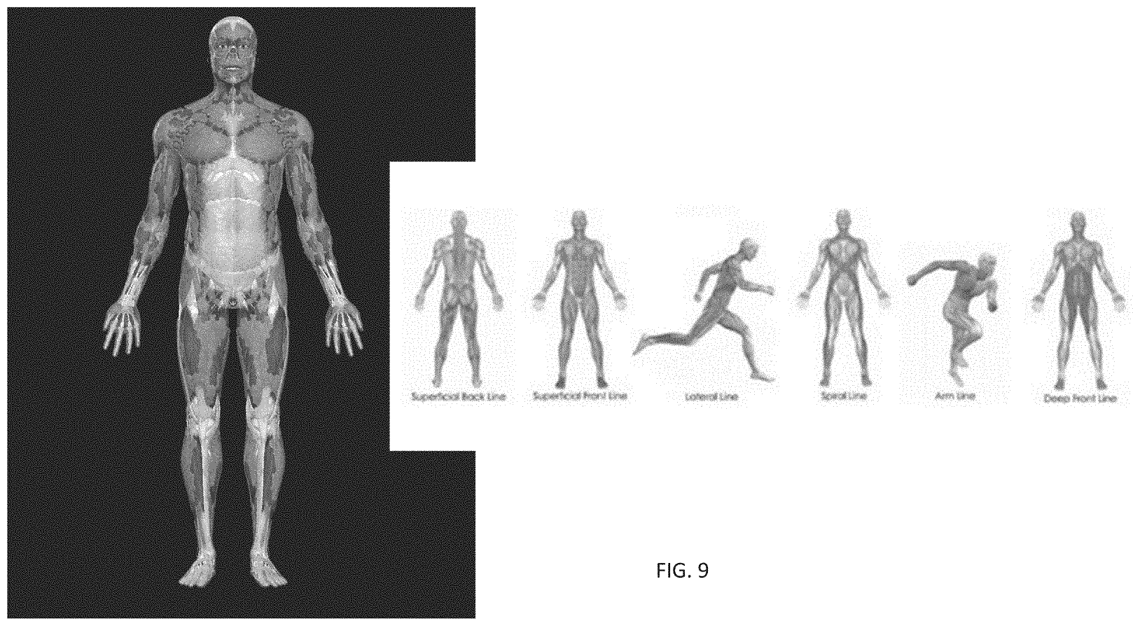

FIG. 21 shows an image of interstitial tissue, in which the connective tissue has fused together in a dense, stiff form to the dermis. In contrast, the image in FIG. 2J shows tissue that has been restored, illustrating an upper epidermal layer 240, a loose connective tissue 250 (dark layer between two light/white layers), and a second layer of dense connective tissue 260 wrapped around an internal muscle. The methods described herein are suitably used to add the desires or required volume of fluid back to the interstitial tissue so as to restore these layers and the “channel” of lose connective tissue 250. Thus, the methods described herein provide a mechanism to return scar tissue back to native muscle and healthy tissue. Testoration of the distinguishable channel in FIG. 21 and FIG. 2J allows for free flow of nutrients, ions, proteins and lymph (interstitium is where the lymph system resides). By restoring this channel, the techniques described herein are creating an environment for healing by allowing the body's “highway” to work properly and tissues to glide properly.

The use of the artificial intelligence (AI) methods described herein and in U.S. Patent Application No. 62/958,430 allow for the determination of the volume of fluid that should be returned to injured interstitial tissue to return it to a healthy state. For example, using the AI methods described herein and in U.S. 62/958,430, the interstitial tissue can be “scored” which provides a measure of the density of the tissue, and conversely, the amount of volume that should be added to the tissue to return it to a healthy state. Following and during the regenerative methods of treating described herein, the tissue is scored using the AI methods to provide a real-time (or near real-time) measure of the pliability of the tissue (Pliability Score), as well as the desired or required volume of fluid to add to the tissue to provide proper restoration of function, elimination of stiffness and pain, and the desired patient outcome. Suitably, the volume that is added back to a particular location of a patient is on the order of about 1 mL to about 500 mL, more suitably about 5 mL to about 400 mL, about 5 mL to about 300 mL, about 5 mL to about 200 mL, about 5 mL to about 150 mL, or less than about 200 mL, or about 150 mL, or less than about 150 mL, etc. Larger volumes can also be utilized, or separate section of the tissue can be restored using a combined number of injections of fluid volume.

FIG. 2L shows a scan of a soft tissue area with a Pliability Score of 29.33%, indicating the presence of calcifications and/or scarred or damaged tissue. FIG. 2K shows the same tissue following restoration using the methods described herein, now demonstrating a Pliability Score of 45.70%, illustrating a more organize tissue, with higher volume, and more healthy structure. Additional results are shown in FIGS. 2M and 2N.

The methods described herein further include introducing a probe into the subepidermal tissue at the site of the entrapped nerve to begin the process of releasing the entrapped nerve, and restructuring and restoring the tissue. FIGS. 3A-3C show exemplary probes that can be used in the methods described herein. In such methods, a “probe” refers to a thin, suitably metallic, plastic, or otherwise biologically inert structure, that can be inserted below the skin surface and directed to the site of the entrapped nerve. Exemplary probes include various needle probes, such as needle probe 300 in FIG. 3A, which includes a hub 304, suitably for connecting the needle to a syringe, luer-lock connection, tubing, etc. Needle probe 300 also includes a shaft 302, which forms the length and structural element of the probe, as well as one or more lumens 306 that allow for fluid to exit from the needle into the tissue. Lumens 306 can take the form of a hole at the tip of the needle (suitably in the form of a beveled opening), or one or more fenestrated holes (slits, apertures, holes, cracks, etc.), to allow for delivery of the liquid composition. As shown in FIG. 3B, fenestrated holes 306 can take the form of slits in the sides of the needle, or small holes, as in FIG. 3C. Suitably, the length of a probe, including a needle probe, is on the order of about 0.4-10 cm, more suitably about 1-5 cm in length, or about 2-4 cm in length. Exemplary fenestrated needle probes are disclosed in U.S. Patent Application No. 62/787,431, filed Jan. 2, 2019, and entitled “Fenestrated Needle for Subcutaneous Use,” the disclosure of which is incorporated by reference herein in its entirety. Suitably, needle probes for use in the methods described herein include radially offset fenestrations beginning at a minimum of about 0.060 inches form the base of the tip to a maximum of about maximum of 0.150 inches from the tip. Suitably the needle probes include a three decimal tolerance of 0.005 in horizontal and vertical hole positioning. The holes are suitably completely through the material to allow for fluid flow. The hole positions can be radial and can also be slotted for maximum diffusion of fluid. Suitably, the lumens 306 are positioned about 0.07-0.10 inches from the tip 310 of the needle 302, and are spaced apart about 0.10 inches. Exemplary positions for lumens 306 are shown in FIG. 3D, though other configurations and spacings can also be used. Suitably, lumens 306 as shown in FIG. 3D have a diameter of about 0.01 in to about 0.05 in, more suitably about 0.02 in to about 0.04 in, or about 0.02 in, about 0.03 in or about 0.04 in.

Suitable needle probe materials include highly conductive metals, such as a highly resonant metal alloy (kansa), and a bell metal (a form of bronze with a higher tin content, usually in approximately a 4:1 ratio of copper to tin (typically, 78% copper, 22% tin by mass)). Additional metals for use in the needle probes include, for example, surgical stainless steel, heat-treatable stainless steel, carbon steel, etc. Typically the gauge of the needle probes used in the methods described herein are between 7 gauge to 33 gauge, suitably 7-10 gauge, and in general are 8 gauge needles. Additional needles include gauge 22 needs, which have an end to end length of about 1.00 inches, an inner radius of about 0.70 mm and an outer radius of about 0.41 mm.

In suitable embodiments, a pre-loaded needle probe is provided, in which the desired amount of fluid for a procedure (e.g., about 10 mL to about 50 mL, or up to about 100 mL), is loaded into a syringe and attached to a fenestrated needle. This pre-loading minimizes the need to withdraw the needle and add fluid for the procedure, thus decreasing the time required for the procedure and also insuring accurate fluid deposition and minimizing of patient discomfort.

Biosculpting tips can also be utilized on the ends of the fenestrated needles to help manipulate the interstitial tissue and move it into the proper tissue planes See for example, FIGS. 3E-3H.

FIG. 4A shows the introduction of a needle probe 300 into the subepidermal tissue 400, at or near the site of one or more areas of fibrosis 104, as well as an entrapped nerve (not visible in this image). Suitably this introduction occurs following the application of a numbing agent and/or a local anesthetic to the area where the needle is to be inserted.

The methods of treatment described herein further include manipulating the subepidermal tissue and/or the entrapped nerve with the probe to aid in releasing or relieving the entrapped nerve, and restructuring the area of fibrosis 104. FIG. 4B shows movement of the needle probe 300 within the subepidermal tissue 400 near the areas of fibrosis 104, resulting in breaking up of scar tissue 404, as the dense, connective tissue is freed and begins to restructure. Movement of the needle probe 300 within the subepidermal tissue 400 during this process can include various sliding back and forth, as well as vibration and/or separation of the tissue via the needle to assist in the restructuring and reorganizing of the scarred tissue. As described herein, needle probe 300 is suitably guided using ultrasound imaging to provide a clear picture of where the needle probe 300 is located within the tissue.

Along with insertion, movement, and vibration of the probe, the methods of treatment described herein suitably include injecting a liquid composition into the subepidermal tissue via the probe at the site of the entrapped nerve. As described herein, it has been determined that hydration of the subepidermal tissue, including scarred or fibrotic connective tissue, can be restored and restructured with the addition of a liquid composition to hydrate the tissue. FIG. 4C shows the results of such a method, in which a nerve 406 has now been freed from the fibrotic tissue 104, and the surrounding subepidermal tissue returns to a more natural, pre-damaged state, in which the cutaneous tissue is more layered in structure, able to move in response to movements of the patient. As described herein, the combination of the insertion, movement and vibration of the probe, and/or the injection (pressure) of a liquid composition into the subepidermal tissue results in a biological fracking, in which tissue is freed and returned to a normal, pre-damaged or pre-scarred state. Nerves are put back into the correct plane through remodeling and restoration of the tissue planes. Dense scarred tissue is broken up and returned to laminar and a native skeletal state.

FIGS. 5A-5B shows the results of an additional remodeling procedure as described herein. In ultrasound image 500, needle probe 300 can be seen inserted into fibrotic tissue 104. In FIG. 5B, following one or more of insertion, movement, and vibration of the probe, along with injecting the liquid composition described herein, the fibrotic tissue 104 can be seen in ultrasound image 502 to be broken up, rehydrated and restructured, illustrated by a less dense image and more spread apart image. Rehydration of the tissue is another unexpected and surprising result of the methods described herein, which allows the tissues to be revitalized.

The liquid composition that is injected into the subepidermal tissue suitably comprises one or more regenerative proteins, one or more cellular proteins, one or more classes of cells, and a buffer. As used herein a “regenerative protein” refers to a protein or amino acid that is involved in the proliferation and differentiation of one or more cell types. “Cellular protein” refers to any protein found within one or more cell types, and can include proteins involved in cellular proliferation, cellular senescence, cell signaling, etc. Exemplary cells that can be included in the liquid compositions include various stem cells, including pluripotent stem cells, etc. Additional healing agents that can be added to the liquid compositions for use in the methods described herein include, for example, exosomes, various amino acids, peptides, and derivatives thereof, various vitamins, growth factors, etc. Exemplary buffers that can be utilized in the liquid compositions include, but are not limited to, saline buffers, phosphate buffers, acetate buffers, citrate buffers, etc., as well as various electrolyte compositions including those that mimic human physiological plasma, such as Plasma-Lyte. Additional components of the liquid compositions used herein can include various sugars, including glucose, dextrose, lactose, etc., as well as various salts, and other components typically used in physiological solutions. Amounts of the various sugars, including dextrose, are suitably in the range of about 1-10%, or about 5%. The liquid compositions can also include a mild anesthetic to help in the reduction of pain at the probe insertion site.

Suitably, the liquid compositions described herein include one or more placental proteins. “Placental proteins” include proteins suitably found in the placenta and/or amniotic fluid of a female mammal, and can be naturally derived or synthetic. Exemplary placental proteins that can be utilized in the liquid compositions described herein include one or more of the following proteins:
Basic fibroblast growth factor (bFGF);
Epidermal growth factor (EGF);
Granulocyte colony-stimulating factor (GCSF);
Platelet-derived growth factor (PDGF-AA);
Platelet-derived growth factor (PDGF-BB);
Placental growth factor (PLGF);
Transforming growth factor alpha (TGF-alpha);
Transforming growth factor beta 1 (TGF-B1);
interleukin 4 (IL-4);
interleukin 6 (IL-6);
interleukin 8 (IL-8);
interleukin 10 (IL-10);
Tissue inhibitor of metalloproteinase (TIMP-1);
Tissue inhibitor of metalloproteinase (TIMP-2);
Tissue inhibitor of metalloproteinase (TIMP-4);
Growth Differentiation Factor (GDF-15);
Granulocyte macrophage colony-stimulating factor (GM-CSF);
Interferon gamma (IFN-γ);
Interleukin 1 alpha (IL1-alpha);
Interleukin 1 Beta (IL1-β);
Interleukin 1 receptor antagonist (IL-1ra);
Interleukin 5 (IL-5);
Interleukin 7 (IL-7);
Interleukin 12 p40 (IL-12p40);
Interleukin 12 p70 (IL-12p70);
Interleukin 15 (IL-15);
Interleukin 17 (IL-17);
Interleukin 16 (IL-16);
Macrophage colony-stimulating factor (MCSF);
Osteoprotegerin (OPG);
B lymphocyte chemoattractant (CXCL13) (BLC);
Chemokine ligand 1 (CCL1) (I-309);
Eotaxin-2;
Monocyte chemotactic protein 1 (CCL2) (MCP-1);
Monokine induced by gamma interferon (CXCL9) (MIG);
Macrophage inflammatory protein 1 alpha (CCL3) (MIP-1α);
Macrophage inflammatory protein 1 beta (CCL4) (MIP-1β);
Macrophage inflammatory protein 1D (MIP-5, CCL15) (MIP-1d);
Regulated on activation, normal T cell expressed and secreted (CCL5) (RANTES);
Brain-derived neurotrophic factor (BDNF);
Bone morphogenetic protein 5 (BMP-5);
Endocrine gland-derived vascular endothelial growth factor (EG-VEGF);
Fibroblast growth factor 4 (FGF-4);
Keratinocyte growth factor (FGF-7);
Growth hormone (GH);
Insulin-like growth factor (IGF-I);
Insulin-like growth factor binding protein-1 (IGFBP-1);
Insulin-like growth factor binding protein-2 (IGFBP-2);
Insulin-like growth factor binding protein-3 (IGFBP-3);
Insulin-like growth factor binding protein-4 (IGFBP-4); and
Insulin-like growth factor binding protein-6 (IGFBP-6).

Exemplary amounts of the placental proteins for use in the liquid compositions include those disclosed in U.S. Published Patent Application No. 2019-0224277, entitled “Bio-Mimetic Formulation,” the disclosure of which is incorporated by reference herein in its entirety, and particular for the amounts and ratios of the placental proteins described therein. For example, the amounts of the placental proteins for use in the liquid compositions include:
Basic fibroblast growth factor (bFGF) at about 4440 (pg/mL) to about 44400 (pg/mL);
Epidermal growth factor (EGF) at about 16.4 (pg/mL) to about 164 (pg/mL);
Granulocyte colony-stimulating factor (GCSF) at about 144 (pg/mL) to about 1440 (pg/mL);
Platelet-derived growth factor (PDGF-AA) at about 34327 (pg/mL) to about 343270 (pg/mL);
Platelet-derived growth factor (PDGF-BB) at about 106 (pg/mL) to about 1060 (pg/mL);
Placental growth factor (PLGF) at about 370 (pg/mL) to about 3700 (pg/mL);
Transforming growth factor alpha (TGF-alpha) at about 3.4 (pg/mL) to about 34 (pg/mL);
Transforming growth factor beta 1 (TGF-B1) at about 1180 (pg/mL) to about 11800 (pg/mL);
interleukin 4 (IL-4) at about 2.2 (pg/mL) to about 22 (pg/mL);
interleukin 6 (IL-6) at about 74 (pg/mL) to about 740 (pg/mL);
interleukin 8 (IL-8) at about 2875 (pg/mL) to about 28750 (pg/mL);
interleukin 10 (IL-10) at about 4.1 (pg/mL) to about 41 (pg/mL);
Tissue inhibitor of metalloproteinase (TIMP-1) at about 16630 (pg/mL) to about 166300 (pg/mL);
Tissue inhibitor of metalloproteinase (TIMP-2) at about 2960 (pg/mL) to about 29600 (pg/mL);
Tissue inhibitor of metalloproteinase (TIMP-4) at about 111 (pg/mL) to about 1110 (pg/mL);
Growth Differentiation Factor (GDF-15) at about 81.25 (pg/mL) to about 812.5 (pg/mL);
Granulocyte macrophage colony-stimulating factor (GM-CSF) at about 0.21 (pg/mL) to about 2.1 (pg/mL);
Interferon gamma (IFNγ at about 2.75 (pg/mL) to about 27.5 (pg/mL);
Interleukin 1 alpha (IL1-alpha) at about 12.5 (pg/mL) to about 125 (pg/mL);
Interleukin 1 Beta (IFN-β at about 27.8 (pg/mL) to about 278 (pg/mL);
Interleukin 1 receptor antagonist (IL-1ra) at about 78.5 (pg/mL) to about 785 (pg/mL);
Interleukin 5 (IL-5) at about 2.88 (pg/mL) to about 28.8 (pg/mL);
Interleukin 7 (IL-7) at about 1.37 (pg/mL) to about 13.7 (pg/mL);
Interleukin 12 p40 (IL-12p40) at about 9.55 (pg/mL) to about 95.5 (pg/mL);
Interleukin 12 p70 (IL-12p70) at about 0.79 (pg/mL) to about 7.9 (pg/mL);
Interleukin 15 (IL-15) at about 1.35 (pg/mL) to about 13.5 (pg/mL);
Interleukin 17 (IL-17) at about 1.1 (pg/mL) to about 11 (pg/mL);
Interleukin 16 (IL-16) at about 34.4 (pg/mL) to about 344(pg/mL);
Macrophage colony-stimulating factor (MCSF) at about 4.3 (pg/mL) to about 43 (pg/mL);
Osteoprotegerin (OPG) at about 319.69 (pg/mL) to about 3196.9 (pg/mL);
B lymphocyte chemoattractant (CXCL13) (BLC) at about 60 (pg/mL) to about 600 (pg/mL);
Chemokine ligand 1 (CCL1) (I-309) at about 4 (pg/mL) to about 40 (pg/mL);
Eotaxin-2 at about 0.13 (pg/mL) to about 1.3 (pg/mL);
Monocyte chemotactic protein 1 (CCL2) (MCP-1) at about 76.95 (pg/mL) to about 769.5 (pg/mL);
Monokine induced by gamma interferon (CXCL9) (MIG) at about 780 (pg/mL) to about 7800 (pg/mL);
Macrophage inflammatory protein 1 alpha (CCL3) (MIP-1α at about 13.08 (pg/mL) to about 130.8 (pg/mL);
Macrophage inflammatory protein 1 beta (CCL4) (MIP-113 at about 6.56 (pg/mL) to about 65.6 (pg/mL);
Macrophage inflammatory protein 1D (MIP-5, CCL15) (MIP-1d) at about 4.3 (pg/mL) to about 43 (pg/mL);
Regulated on activation, normal T cell expressed and secreted (CCL5) (RANTES) at about 203 (pg/mL) to about 2030 (pg/mL);
Brain-derived neurotrophic factor (BDNF) at about 45.03 (pg/mL) to about 450.3 (pg/mL);
Bone morphogenetic protein 5 (BMP-5) at about 90.55 (pg/mL) to about 905.5 (pg/mL);
Endocrine gland-derived vascular endothelial growth factor (EG-VEGF) at about 496.68 (pg/mL) to about 4966.8 (pg/mL);
Fibroblast growth factor 4 (FGF-4) at about 353.37 (pg/mL) to about 3533.7 (pg/mL);
Keratinocyte growth factor (FGF-7) at about 46.7 (pg/mL) to about 467 (pg/mL);
Growth hormone (GH) at about 114.23 (pg/mL) to about 1142.3 (pg/mL);
Insulin-like growth factor (IGF-I) at about 27.65 (pg/mL) to about 276.5 (pg/mL);
Insulin-like growth factor binding protein-1 (IGFBP-1) at about 353.51 (pg/mL) to about 3535.1 (pg/mL);
Insulin-like growth factor binding protein-2 (IGFBP-2) at about 1072.52 (pg/mL) to about 10725.2 (pg/mL);
Insulin-like growth factor binding protein-3 (IGFBP-3) at about 7701.19 (pg/mL) to about 77011.9 (pg/mL);
Insulin-like growth factor binding protein-4 (IGFBP-4) at about 2954.34 (pg/mL) to about 29543.4 (pg/mL); and
Insulin-like growth factor binding protein-6 (IGFBP-6) at about 5162.16 (pg/mL) to about 51621.6 (pg/mL).

In exemplary embodiments, a subgroup of the placental proteins can be used, including for example:
Angiogenin
Chondroitin
Collagens I, III, IV, V, VI, VII
b-defensins
EGF
Elafin
Elastin
Fibronectin
Heparin Sulfate
Hyaluronic Acid
FGF
HGF
IFNγ
IGF-1
IL-4
KGF
Laminin
MMPs and TIMPs
Nidogen
PDGF
PIGF
SLPI
TGF-α
TGF-β
TNF
VEGF
Vitronectin

In further embodiments, the placental proteins used in the liquid compositions include the following at the concentration range provided:

TABLE 2
Exemplary Concentrations of Placental
Proteins in Liquid Composition

| Bioactive Factor | Concentration (pg/mL) |
|---|---|
| PDGF-AA | 71.4 ± 21.4 |
| PDGF-BB | 45.2 ± 14.5 |
| bFGF | 165.5 ± 53.2 |
| EGF | 298.8 ± 108.0 |
| KGF | 9.16 ± 3.00 |
| PIGF | 12.0 ± 2.81 |
| IL-4 | 230.7 ± 77.6 |
| TGF-β1 | 897.7 ± 446.1 |
| TGF-β3 | 506.4 ± 95.8 |
| VEGF | N.D. |
| TIMP-1 | 7663 ± 2869 |
| TIMP-2 | 7188 ± 1342 |

The volume of the liquid composition that is injected into the subepidermal tissue is suitably in the range of about 20-200 mL, more suitably about 50-150 mL, for each treatment session. This amount can also be re-administered, as described herein, at follow-up visits with the patient.

In exemplary embodiments, the methods of treatment described herein can include the movement by the patient causing movement of the subepidermal tissue, during or coinciding any one or more of the visualizing, vibrating and/or locating steps described herein. By having the patient move the subepidermal tissue being treated, the underlying scarred tissue and/or entrapped nerves can be better visualized. Kinetics of tissue planes are assessed by having the patient move the local tissue (e.g. flexing arm, extending leg, etc). Tissue is visualized under ultrasound and also via elastography (red is healthy tissue, white is stiff, dense tissue). Generally, this movement by the patient relates directly to movements that cause the patient discomfort or pain, including for example, bending one or more joints (e.g., bending at the knees, or back, neck movements, shoulder, elbow and arm movements, hand and finger movements, etc.) Hypokinetic areas are indicative of suboptimal physiology (stiffness, pain, swelling due to lymphatic outflow obstruction). This dynamic method can be used with the ultrasound imaging, resulting in a dynamic imaging including a dynamic ultrasound imaging or visualization to further elucidate the entrapped nerve(s) and/or the scarred tissue.

As described herein, in further embodiments the mesoderm-derived tissue that is treated results in remodeling of surface tissue. For example, scar tissue can be remodeled below the skin, resulting in the changes in morphology of the skin covering the scar tissue. As shown in FIGS. 6A-6B, scar tissue below an incision (FIG. 6A) can be remodeled, resulting in dramatic changes in the tightness and appearance of surface skin (FIG. 6B).

FIG. 7A shows surface skin wrinkles after a failed neck lift surgery, and FIG. 7B the removal of neck wrinkles following a restructuring of the underlying tissues using the methods described herein.

FIG. 7C shows pre and FIG. 7D shows post-images of a patient's skin following treatment of the underlying tissues using the methods described herein. The patient's skin is rejuvenated, nasolabial folds are reduced, and the nose is thinned.

FIG. 7E and FIG. 7F show before and after treatment of the tissues below the skin, demonstrating the removal of the patient's forehead wrinkles.

Additional results of the methods of treatment described herein for rejuvenating mesenchymal tissue are provided in FIGS. 8A-8F.

As shown in FIG. 8A, treatment of the underlying scalp tissue of a man experiencing acute alopecia demonstrated hair regrowth in 30 days (FIG. 8B).

FIGS. 8C and 8D show before and after treatment of a patient's scar from removal of a melanoma.

FIGS. 8E and 8F show before and after treatment of a patient's arm, demonstrating removal of arm wrinkles, skin rejuvenation and lifting.

As shown in FIG. 9, connective tissue can be found throughout the body, providing significantly diverse areas where the methods and techniques described herein can be utilized. FIG. 9 shows the six (6) fascial planes, illustrating that connective tissue covers the body from head to toe, allowing the methods and techniques described herein anywhere connective tissue can be found. It has been surprisingly discovered that treating across these 6 fascial planes augments the pain relieving/healing results, as everything is connected, and thus knee pain may come from an anteriorly rotated hip, for example.

The methods of treatment described herein suitably further include additional, post-injection techniques, that are used to dissipate the liquid composition within the subepidermal tissue. That is, following the injection of the liquid composition into the tissue of the patient and the freeing of the entrapped nerve(s), in order to facilitate movement of the liquid composition and enhance hydration, and thus restructuring and healing of the scarred tissue, various techniques are suitably employed. These techniques can include manual manipulation of the treated area via massage by hand, or by use of a percussion or other massaging device. Additional techniques include application of a sound wave, for example from an ultrasound or other device, including at a sound frequency of about 20-2000 MHz, more suitably about 20-200 MHz, or about 50-200 MHz, etc. Further techniques include the use of a pulsed electromagnetic field, for example from of a transmission of a multi-dimensionally configured signal (waveform).

These additional post-treatment techniques to dissipate the liquid composition within the tissue also provide a mechanism for activating telocytes and telomerase to support the regeneration of healthy tissue and modulate scarring and inflammation. Combinations of physical input with restoration of connective tissue (e.g., via a electromagnetic field) allows for maximizing the correct cellular orientation to instruct the anatomy to repair.

In additional embodiments, provide herein is a method for treating a mesoderm-derived tissue in a patient. The method suitably includes visualizing the mesoderm-derived tissue beneath a skin surface of the patient via dynamic ultrasound imaging and real-time image analysis. The method further includes vibrating the connective tissue beneath the skin surface, and locating one or more entrapped nerves within the connective tissue. A needle probe is suitably introduced into the connective tissue at the site of the entrapped nerve. The method suitably includes manipulating the tissue and/or the entrapped nerve with the needle probe. Suitably, a liquid composition is injected into the connective tissue via the needle probe, the liquid composition comprising one or more regenerative proteins and a buffer.

As described herein, in embodiments, the mesoderm-derived tissue is a connective tissue, and is suitably located in the patient's neck, back, knees, hips, shoulders, elbows, feet, ankles, toes, hands, wrists, and/or fingers. Exemplary components of the liquid composition for use in the methods are described herein, and suitably include dextrose, plasma-lyte, and one or more placental proteins, including those described herein.

Exemplary ultrasound imaging frequencies are described herein, and include operating frequencies of about 4-100 MHz. The connective tissue is suitably located within about 10 cm from the skin surface of the patient, but as described herein, can be at a greater depth depending on the physical characteristics of the patient (e.g., patient weight and body mass).

As described herein, the vibrating to assist in the visualizing is suitably an acoustic radiation force impulse, including a shear wave, or a palpitation. Exemplary needle probes for use in the methods, including fenestrated needle probes, are described herein.

Methods for manipulating the entrapped nerve includes vibration and/or separation of the tissue via the needle probe, as described herein.

As noted herein, suitably the methods of treatment further include dissipating the liquid composition within the connective tissue via one or more of manual manipulation, percussion massaging, application of a sound wave, and application of a pulsed electromagnetic field.

The methods of treatment described herein are used to help restructure and rejuvenate scarred tissue, including connective tissue. It has also been determined that movement of the connective tissue by the patient following the various treatment procedures described herein can help aid in re-structuring the connective tissue. This movement includes mild exercise and specifically stretching and expanding the areas where treatment has taken place, to help realign the connective tissues and speed healing.

While the methods described herein can be performed a single time at a single site on a patient, the procedures can also be repeated as needed to further aid in restructuring. In addition, the methods described herein can further include re-administering the liquid composition comprising regenerative proteins to the connective tissue at various time intervals. Suitably, the liquid compositions are re-administered at about 7-120 days following the initial administration, more suitably at about 14-90 days after the initial administering. In exemplary embodiments, the re-administration occurs at about ever 7 days, about every 14 days, every 21 days, etc., including every 14-90 days. Suitably, the re-administration occurs about every 14-90 days, for a period of 1-12 months, including about 1-6 months. In embodiments, patients are treated every 14 days, for about 1-10 times, or about 1-6 times, over a period of about 1-6 months, more suitably about 3-4 months.

The methods of treatment described herein have been shown to restore movement, eliminate stiffness, and reduce or eliminate pain in the joints and connective tissue of several patients, including professional athletes that have endured numerous years of stress and high impact workouts during their careers.

In further embodiments, provided herein is a kit for carrying out treatment of connective tissue of a patient. Suitably the kit includes a liquid composition comprising dextrose, plasma-lyte and one or more placental proteins selected from the group consisting of:
Basic fibroblast growth factor (bFGF);
Epidermal growth factor (EGF);
Granulocyte colony-stimulating factor (GCSF);
Platelet-derived growth factor (PDGF-AA);
Platelet-derived growth factor (PDGF-BB);
Placental growth factor (PLGF);
Transforming growth factor alpha (TGF-alpha);
Transforming growth factor beta 1 (TGF-B1);
interleukin 4 (IL-4);
interleukin 6 (IL-6);
interleukin 8 (IL-8);
interleukin 10 (IL-10);
Tissue inhibitor of metalloproteinase (TIMP-1);
Tissue inhibitor of metalloproteinase (TIMP-2);
Tissue inhibitor of metalloproteinase (TIMP-4);
Growth Differentiation Factor (GDF-15);
Granulocyte macrophage colony-stimulating factor (GM-CSF);
Interferon gamma (IFN-γ);
Interleukin 1 alpha (IL1-alpha);
Interleukin 1 Beta (IL1-β);
Interleukin 1 receptor antagonist (IL-1ra);
Interleukin 5 (IL-5);
Interleukin 7 (IL-7);
Interleukin 12 p40 (IL-12p40);
Interleukin 12 p70 (IL-12p70);
Interleukin 15 (IL-15);
Interleukin 17 (IL-17);
Interleukin 16 (IL-16);
Macrophage colony-stimulating factor (MCSF);
Osteoprotegerin (OPG);
B lymphocyte chemoattractant (CXCL13) (BLC);
Chemokine ligand 1 (CCL1) (I-309);
Eotaxin-2;
Monocyte chemotactic protein 1 (CCL2) (MCP-1);
Monokine induced by gamma interferon (CXCL9) (MIG);
Macrophage inflammatory protein 1 alpha (CCL3) (MIP-1α);
Macrophage inflammatory protein 1 beta (CCL4) (MIP-1β);
Macrophage inflammatory protein 1D (MIP-5, CCL15) (MIP-1d);
Regulated on activation, normal T cell expressed and secreted (CCL5) (RANTES);
Brain-derived neurotrophic factor (BDNF);
Bone morphogenetic protein 5 (BMP-5);
Endocrine gland-derived vascular endothelial growth factor (EG-VEGF);
Fibroblast growth factor 4 (FGF-4);
Keratinocyte growth factor (FGF-7);
Growth hormone (GH);
Insulin-like growth factor (IGF-I);
Insulin-like growth factor binding protein-1 (IGFBP-1);
Insulin-like growth factor binding protein-2 (IGFBP-2);
Insulin-like growth factor binding protein-3 (IGFBP-3);
Insulin-like growth factor binding protein-4 (IGFBP-4); and
Insulin-like growth factor binding protein-6 (IGFBP-6).

Exemplary amounts of these placental proteins and preferred proteins are described herein.

The kit suitably further includes a needle probe for injecting the liquid composition into the connective tissue and for manipulating an entrapped nerve, as described herein. In embodiments, the kit further includes instructions for carrying out the method of treatment of the connective tissue, in accordance with embodiments described herein.

Various containers, etc., for holding the components of the kit are known in the art and include, for example, various syringes, bottles, boxes, wraps, bags, etc.

As described herein, in embodiments, the needle probe includes one or more fenestrated holes to allow for delivery of the liquid composition.

The kits described herein can also further include one or more instruments for manual manipulation, percussion massaging, application of a sound wave, or application of a pulsed electromagnetic field, to the connective tissue. Such instruments are suitably used after finishing the treatment methods described herein to dissipate the liquid composition within the tissue of the patient to aid in healing and reducing of swelling.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

While various embodiments have been described above, it should be understood that they have been presented only as illustrations and examples of the present technology, and not by way of limitation. It will be apparent to persons skilled in the relevant art that various changes in form and detail can be made therein without departing from the spirit and scope of the present technology. Thus, the breadth and scope of the present technology should not be limited by any of the above-described embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

1. A method for treating a subepidermal tissue in a patient, comprising:
a. visualizing the tissue beneath a skin surface of the patient;
b. vibrating the tissue beneath the skin surface;
c. locating one or more entrapped nerves within the subepidermal tissue;
d. introducing a probe into the subepidermal tissue at the site of the entrapped nerve;
e. manipulating the subepidermal tissue and/or the entrapped nerve with the probe; and
f injecting a liquid composition into the subepidermal tissue via the probe at the site of the entrapped nerve.

2. The method of claim 1, wherein the subepidermal tissue is a mesoderm-derived tissue.

3. The method of claim 2, wherein the mesoderm-derived tissue is a connective tissue, including a connective tissue located in the patient's neck, back, knees, hips, shoulders, elbows, feet, ankles, toes, hands, wrists, and/or fingers.

4. (canceled)

5. The method of claim 1, wherein the liquid composition comprises one or more regenerative proteins or cells, and a buffer.

6. The method of claim 1, wherein the visualizing comprises ultrasound imaging at a frequency of about 4-100 MHz.

7. (canceled)

8. (canceled)

9. The method of claim 1, wherein the vibrating is an acoustic radiation force impulse, a shear wave, or a palpitation.

10. (canceled)

11. The method of claim 1, wherein the probe is a needle probe, or a needle probe including one or more fenestrated holes to allow for delivery of the liquid composition.

12. (canceled)

13. The method of claim 1, wherein the manipulating the entrapped nerve includes vibration and/or separation of the tissue via the needle.

14. The method of claim 1, wherein the visualizing, vibrating and/or locating coincide with movement by the patient causing movement of the subepidermal tissue.

15. The method of claim 1, wherein the visualizing includes real-time image analysis to locate the entrapped nerves and abnormal tissue.

16. The method of claim 1, further comprising dissipating the liquid composition within the tissue via one or more of manual manipulation, percussion massaging, application of a sound wave, and application of a pulsed electromagnetic field.

17. A method for treating a mesoderm-derived tissue in a patient, comprising:
a. visualizing the mesoderm-derived tissue beneath a skin surface of the patient via dynamic ultrasound imaging and real-time image analysis;
b. vibrating the connective tissue beneath the skin surface;
c. locating one or more entrapped nerves within the connective tissue;
d. introducing a needle probe into the connective tissue at the site of the entrapped nerve;
e. manipulating the tissue and/or the entrapped nerve with the needle probe; and
f. injecting a liquid composition into the connective tissue via the needle probe, the liquid composition comprising one or more regenerative proteins and a buffer.

18. The method of claim 17, wherein the mesoderm-derived tissue is a connective tissue, including a connective tissue located in the patient's neck, back, knees, hips, shoulders, elbows, feet, ankles, toes, hands, wrists, and/or fingers.

19. (canceled)

20. The method of claim 17, wherein the liquid composition comprises dextrose, plasma-lyte and one or more placental proteins selected from the group consisting of:
Basic fibroblast growth factor (bFGF);
Epidermal growth factor (EGF);
Granulocyte colony-stimulating factor (GCSF);
Platelet-derived growth factor (PDGF-AA);
Platelet-derived growth factor (PDGF-BB);
Placental growth factor (PLGF);
Transforming growth factor alpha (TGF-alpha);
Transforming growth factor beta 1 (TGF-B1);
interleukin 4 (IL-4);
interleukin 6 (IL-6);
interleukin 8 (IL-8);
interleukin 10 (IL-10);
Tissue inhibitor of metalloproteinase (TIMP-1);
Tissue inhibitor of metalloproteinase (TIMP-2);
Tissue inhibitor of metalloproteinase (TIMP-4);
Growth Differentiation Factor (GDF-15);
Granulocyte macrophage colony-stimulating factor (GM-CSF);
Interferon gamma (IFN-γ);
Interleukin 1 alpha (IL1-alpha);
Interleukin 1 Beta (IL1-β);
Interleukin 1 receptor antagonist (IL-1ra);
Interleukin 5 (IL-5);
Interleukin 7 (IL-7);
Interleukin 12 p40 (IL-12p40);
Interleukin 12 p70 (IL-12p70);
Interleukin 15 (IL-15);
Interleukin 17 (IL-17);
Interleukin 16 (IL-16);
Macrophage colony-stimulating factor (MCSF);
Osteoprotegerin (OPG);
B lymphocyte chemoattractant (CXCL13) (BLC);
Chemokine ligand 1 (CCL1) (I-309);
Eotaxin-2;
Monocyte chemotactic protein 1 (CCL2) (MCP-1);
Monokine induced by gamma interferon (CXCL9) (MIG);
Macrophage inflammatory protein 1 alpha (CCL3) (MIP-1α);
Macrophage inflammatory protein 1 beta (CCL4) (MIP-1β);
Macrophage inflammatory protein 1D (MIP-5, CCL15) (MIP-1d);
Regulated on activation, normal T cell expressed and secreted (CCL5) (RANTES);
Brain-derived neurotrophic factor (BDNF);
Bone morphogenetic protein 5 (BMP-5);
Endocrine gland-derived vascular endothelial growth factor (EG-VEGF);
Fibroblast growth factor 4 (FGF-4);
Keratinocyte growth factor (FGF-7);
Growth hormone (GH);
Insulin-like growth factor (IGF-I);
Insulin-like growth factor binding protein-1 (IGFBP-1);
Insulin-like growth factor binding protein-2 (IGFBP-2);
Insulin-like growth factor binding protein-3 (IGFBP-3);
Insulin-like growth factor binding protein-4 (IGFBP-4); and
Insulin-like growth factor binding protein-6 (IGFBP-6).

21. The method of claim 17, wherein the ultrasound imaging operates at a frequency of about 4-100 MHz.

22. (canceled)

23. The method of claim 17, wherein the vibrating is an acoustic radiation force impulse, a shear wave or a palpitation.

24. (canceled)

25. The method of claim 17, wherein the needle probe includes one or more fenestrated holes to allow for delivery of the liquid composition.

26. The method of claim 17, wherein the manipulating the entrapped nerve includes vibration and/or separation of the tissue via the needle.

27. The method of claim 17, further comprising dissipating the liquid composition within the connective tissue via one or more of manual manipulation, percussion massaging, application of a sound wave, and application of a pulsed electromagnetic field, and/or further comprising movement of the connective tissue by the patient to help aid in re-structuring the connective tissue.

28. (canceled)

29. The method of claim 17, further comprising re-administering the composition comprising regenerative proteins to the connective tissue at about 14-90 days after the initial administering.

30. (canceled)

31. (canceled)

32. (canceled)

33. (canceled)
